(19) United States
(12) Patent Application Publication
(10) Pub. No.: US 2023/0266136 A1
(43) Pub. Date: Aug. 24, 2023

(54) INFORMATION PROCESSING DEVICE, INFORMATION PROCESSING METHOD, AND NON-TRANSITORY STORAGE MEDIUM

(71) Applicant: TOYOTA JIDOSHA KABUSHIKI KAISHA, Toyota-shi (JP)

(72) Inventors: Toyokazu NAKASHIMA, Nagoya-shi (JP); Shunsuke SAGARA, Nisshin-shi (JP)

(21) Appl. No.: 18/061,525

(22) Filed: Dec. 5, 2022

(30) Foreign Application Priority Data
Feb. 18, 2022 (JP) 2022-023774

(51) Int. Cl. G01C 21/34; G06V 20/13; G01W 1/10

(52) U.S. Cl. CPC G01C 21/3469; G01C 21/3461; G06V 20/13; G01W 1/10

(57) ABSTRACT

The information processing device acquires first information related to the exhaust gas in a predetermined area at a predetermined timing observed by the artificial satellite. Further, the information processing device acquires the second information regarding the exhaust gas of the vehicle newly generated in the predetermined area after the predetermined timing. Further, the information processing device acquires third information on weather within a predetermined area after a predetermined timing. Then, the information processing device predicts the concentration of the exhaust gas in the predetermined area after the predetermined timing based on the first information, the second information, and the third information.

### CROSS-REFERENCE TO RELATED APPLICATION

This application claims priority to Japanese Patent Application No. 2022-023774 filed on Feb. 18, 2022, incorporated herein by reference in its entirety.

### BACKGROUND

### 1. Technical Field

The present disclosure relates to an information processing device, an information processing method, and a non-transitory storage medium.

### 2. Description of Related Art

Japanese Unexamined Patent Application Publication No. 2021-092396 (JP 2021-092396 A) discloses an environment prediction system. In the environment prediction system disclosed in JP 2021-092396 A, environment data measured by a plurality of moving objects is collected in association with measurement position data of the moving objects. In addition, in the environment prediction system, the measured environment data is corrected based on the measured position data. Then, in the environment prediction system, the spatial environment prediction at the future time is performed based on the corrected environment data and the measured position data.

### SUMMARY

An object of the present disclosure is to enable countermeasures against air pollution.

An information processing device according to a first aspect of the present disclosure includes a control unit. The control unit executes: acquisition of first information about exhaust gas in a predetermined area at a predetermined timing observed by an artificial satellite; acquisition of second information about exhaust gas of a vehicle that is newly generated in the predetermined area at or after the predetermined timing; acquisition of third information about weather in the predetermined area at or after the predetermined timing; and prediction of a concentration of the exhaust gas in the predetermined area at or after the predetermined timing based on the first information, the second information, and the third information.

An information processing method according to a second aspect of the present disclosure is an information processing method executed by a computer, and includes: acquiring first information about exhaust gas in a predetermined area at a predetermined timing observed by an artificial satellite; acquiring second information about exhaust gas of a vehicle that is newly generated in the predetermined area at or after the predetermined timing; acquiring third information about weather in the predetermined area at or after the predetermined timing; and predicting a concentration of the exhaust gas in the predetermined area at or after the predetermined timing based on the first information, the second information, and the third information.

A non-transitory storage medium according to a third aspect of the present disclosure stores instructions executable by one or more processors and causing the one or more processors to perform functions, and the functions include: acquiring first information about exhaust gas in a predetermined area at a predetermined timing observed by an artificial satellite; acquiring second information about exhaust gas of a vehicle that is newly generated in the predetermined area at or after the predetermined timing; acquiring third information about weather in the predetermined area at or after the predetermined timing; and predicting a concentration of the exhaust gas in the predetermined area at or after the predetermined timing based on the first information, the second information, and the third information.

The present disclosure enables countermeasures against air pollution.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram illustrating a schematic configuration of an observation system according to a first embodiment;

FIG. 2 is a diagram illustrating an example of a situation of exhaust gas in a predetermined area at a predetermined timing;

FIG. 3 is a diagram illustrating an example of a situation of exhaust gas in a predetermined area after a predetermined timing;

FIG. 4 is a block diagram schematically illustrating an example of a functional configuration of the prediction server according to the first embodiment;

FIG. 5 is a diagram illustrating an example of a table configuration of observation information held in an observation information database;

FIG. 6 is a diagram illustrating an example of a table configuration of route information held in a route information database;

FIG. 7 is a diagram illustrating an example of a table configuration of new gas information held in a new gas information database;

FIG. 8 is a diagram illustrating an example of a table configuration of weather information held in a weather information database;

FIG. 9 is an example of a table configuration of user information held in a user information database;

FIG. 10 is a flowchart of a generation process;

FIG. 11 is a flowchart of notification processing;

FIG. 12 is a diagram illustrating an example of a situation of exhaust gas in a predetermined area;

FIG. 13 is a flowchart of the detour proposal process.

FIG. 14 is a diagram illustrating a schematic configuration of an observation system according to the third embodiment;

FIG. 15 is a block diagram schematically illustrating an example of a functional configuration of the prediction server according to the third embodiment;

FIG. 16 is a diagram illustrating an example of a table configuration of gas station information held in a gas station information database;

FIG. 17 is a flowchart of the price determination process.

### DETAILED DESCRIPTION OF EMBODIMENTS

An information processing device according to a first aspect of the present disclosure is an information processing device related to prediction of air pollution. Here, it is assumed that a state of air pollution in a predetermined area is observed by an artificial satellite. In this case, since the artificial satellite has the number of days of regression, it is difficult to continuously observe the state of air pollution in a predetermined area in real time by the artificial satellite. Therefore, it is difficult to grasp the state of air pollution in a predetermined area after a predetermined timing.

Therefore, the control unit of the information processing device according to the first aspect of the present disclosure acquires the first information, the second information, and the third information. Here, the first information is information on exhaust gas in a predetermined area at a predetermined timing observed by the artificial satellite. Further, the second information is information related to exhaust gas of the vehicle newly generated within a predetermined area after a predetermined timing. The third information is information on weather within a predetermined area after a predetermined timing.

The control unit of the information processing device can grasp the exhaust gas in a predetermined area at a predetermined timing by acquiring the first information. Further, by acquiring the second information, the control unit can recognize the exhaust gas of the vehicle newly generated in the predetermined area after the predetermined timing. Further, by acquiring the third information, the control unit can grasp the influence of the weather in the predetermined area after the predetermined timing on the exhaust gas in the predetermined area. Therefore, the control unit predicts the concentration of the exhaust gas in the predetermined area after the predetermined timing based on the first information, the second information, and the third information.

As described above, the information processing device according to the first aspect of the present disclosure makes it possible to predict the state of air pollution in a predetermined area after a predetermined timing. As a result, it is possible to take measures against air pollution based on the predicted air pollution situation.

Hereinafter, embodiments of the present disclosure will be described below with reference to the drawings. Unless otherwise specified, dimensions, materials, shapes, relative arrangements, and the like of components described in the present embodiments are not intended to limit the technical scope of the present disclosure to those alone.

### First Embodiment

### System Overview

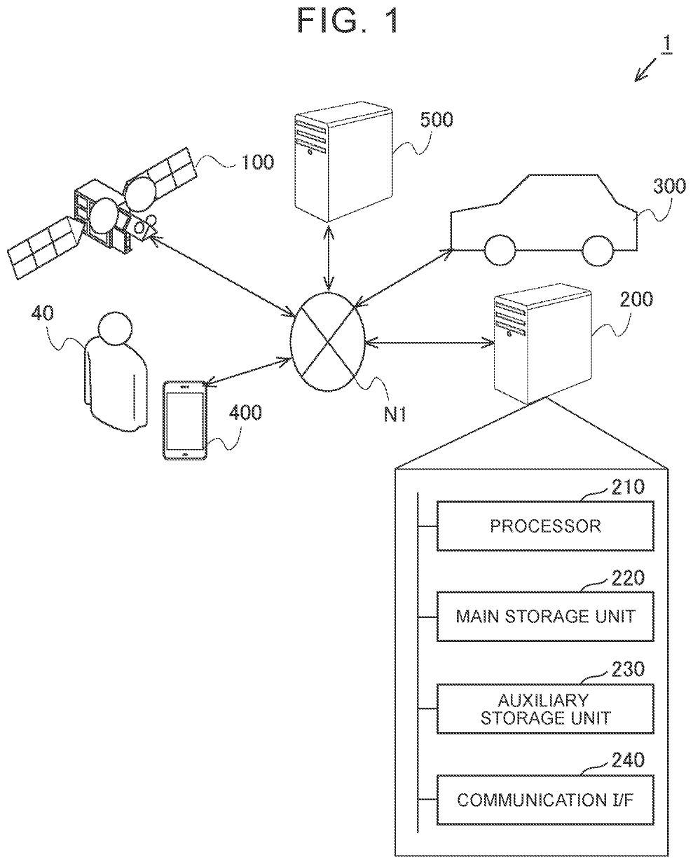

The observation system 1 according to the present embodiment will be described with reference to FIGS. 1 to 3. FIG. 1 is a diagram illustrating a schematic configuration of an observation system 1 according to the present embodiment. The observation system 1 includes an artificial satellite 100, a prediction server 200, a vehicle 300, a user terminal 400, and a weather server 500. In the observation system 1, a satellite 100, a prediction server 200, a plurality of vehicles 300, a user terminal 400, and a weather server 500 are connected to each other via a network N1. The network N1 may be, for example, a worldwide public communication network such as Internet or the like, and a WAN (Wide Area Network) or a telecommunications network such as a cellular network.

### (Satellites)

The satellite 100 is an artificial satellite for observing air pollution. The satellites 100 include, for example, Ozone Monitoring Instrument (OMI) sensors. By using the OMI sensor, the satellite 100 observes an atmospheric concentration of a substance related to the exhaust gas at each point in a predetermined area (hereinafter, may be simply referred to as “concentration”). In the present embodiment, the satellite 100 can measure the concentration of nitrogen oxide as the concentration of the substance related to the exhaust gas. The artificial satellite 100 transmits information on exhaust gas in a predetermined area at a predetermined timing (hereinafter, sometimes referred to as “observation information”) to the prediction server 200 via the network N1. Note that the nitrogen oxide observed by the satellite 100 may contain nitrogen oxide contained in exhaust gas other than the vehicle 300 (for example, exhaust gas of a factory).

In the present embodiment, the satellite 100 observes the concentration of nitrogen oxide as a substance related to the exhaust gas by the OMI sensor. However, the satellite 100 may observe the concentration of a substance other than nitrogen oxides as a substance related to the exhaust gas by the OMI sensor. The satellite 100 may observe, for example, the concentration of sulfur oxide in the atmosphere in a predetermined area by an OMI sensor. Further, the satellite 100 does not necessarily have to observe the concentration of the substance related to the exhaust gas in the atmosphere using the OMI sensor. The satellite 100 may observe the concentration of the substance associated with the exhaust gas using, for example, an optical sensor. The satellites 100 may observe the density of particulate matter such as PM2.5 in the atmosphere, for example, by observing the polarization by an optical sensor. The satellite 100 may also observe the concentration of hydrocarbons in the atmosphere as a substance related to the exhaust gas. The satellite 100 may also observe the concentration of carbon monoxide in the atmosphere as a substance related to the exhaust gas.

### Vehicle

The vehicle 300 is a vehicle that travels in a predetermined area including a predetermined area. When the vehicle 300 travels, exhaust gas is generated. Therefore, air pollution occurs in a predetermined area as the vehicle 300 travels.

The vehicle 300 periodically transmits information including a travel schedule route of the vehicle 300 to the prediction server 200 via the network N1. As a result, the prediction server 200 can recognize the scheduled traveling route of the vehicle 300. The prediction server 200 receives information including the scheduled traveling route of each vehicle 300 from a plurality of vehicles 300 traveling in a predetermined area.

### (Weather Server)

The weather server 500 is a server that holds information on weather within a predetermined area. The weather server 500 transmits information on weather in a predetermined area (hereinafter, may be referred to as “weather information”) to the prediction server 200.

### Server

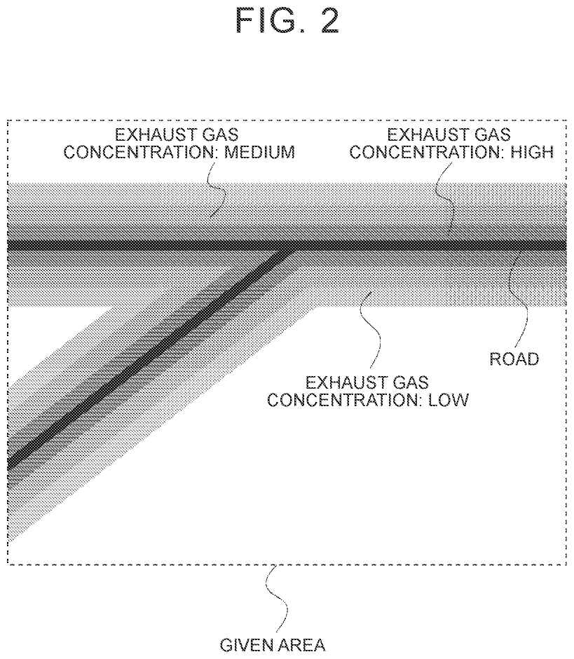

The prediction server 200 is a server related to prediction of air pollution. The prediction server 200 receives the observation information from the artificial satellite 100. Thus, the prediction server 200 can grasp the concentration of the exhaust gas in a predetermined area at a predetermined timing. FIG. 2 is a diagram illustrating an example of a situation of exhaust gas in a predetermined area at a predetermined timing. As shown in FIG. 2, in a predetermined area at a predetermined timing, the concentration of the exhaust gas is high around the road. Further, in the example shown in FIG. 2, the closer to the road, the higher the concentration of the exhaust gas. Here, in the example shown in FIG. 2, the exhaust gas concentration is divided into high, medium, and low levels according to the concentration of nitrogen oxides in the atmosphere.

Here, it is assumed that the artificial satellite 100 is an artificial satellite having a fixed number of regression days. In this case, the satellite 100 can observe the concentration of the exhaust gas (the concentration of nitrogen oxides) in the predetermined area when the satellite 100 is above the predetermined area. Therefore, when the satellite 100 is an artificial satellite having a fixed number of regression days, it is difficult to continuously observe the concentration of the exhaust gas in a predetermined area.

Therefore, the prediction server 200 acquires new gas information related to exhaust gas of the vehicle 300 (hereinafter, may be referred to as “new gas”) newly generated in a predetermined area after a predetermined timing. As a result, the prediction server 200 can recognize the exhaust gas of the vehicle 300 newly generated in the predetermined area after the predetermined timing. Details of a method of acquiring new gas information by the prediction server 200 will be described later.

Further, the prediction server 200 acquires weather information related to weather within a predetermined area after a predetermined timing. The prediction server 200 receives weather information from the weather server 500 via the network N1. The weather information includes information on a wind direction and a wind speed within a predetermined area after a predetermined timing. As a result, the prediction server 200 can grasp the influence of the wind on the exhaust gas in a predetermined area after a predetermined timing.

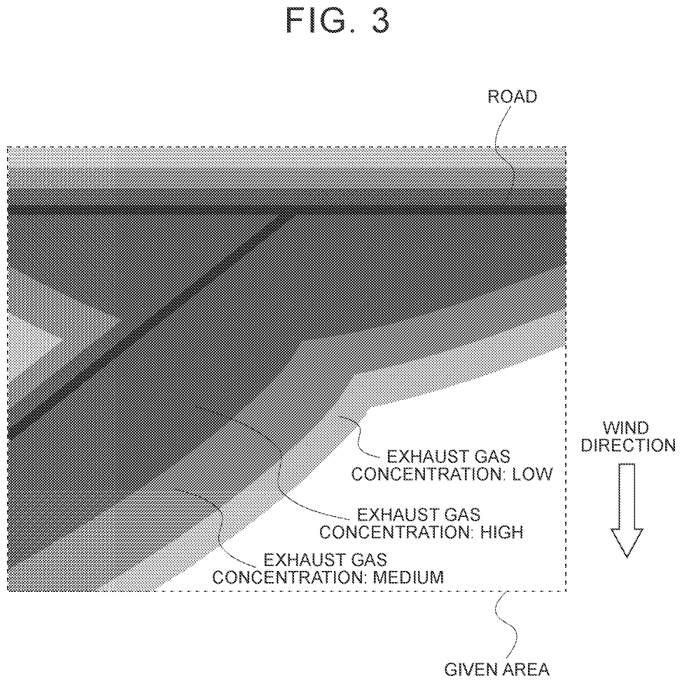

FIG. 3 is a diagram illustrating an example of a situation of exhaust gas in a predetermined area after a predetermined timing. As shown in FIG. 3, in a predetermined area, a wind is blown from above the paper surface toward below the paper surface. In addition, in the predetermined area, exhaust gas of the vehicle 300 is newly generated after the predetermined timing. Therefore, the state of the exhaust gas shown in FIG. 2 is changed as shown in FIG. 3 by the wind blowing in a predetermined area and the newly generated exhaust gas. Therefore, the prediction server 200 predicts the concentration of the exhaust gas in the predetermined area after the predetermined timing based on the observation information, the new gas information, and the weather information. Details of a method of predicting the concentration of the exhaust gas in a predetermined area after a predetermined timing by the prediction server 200 will be described later.

The prediction server 200 includes a computer having a processor 210, a main storage unit 220, an auxiliary storage unit 230, and a communication interface (communication I/F) 240. The processor 210 is, for example, a Central Processing Unit (CPU) or a Digital Signal Processor (DSP). The main storage unit 220 is, for example, a Random Access Memory (RAM). The secondary storage unit 230 is, for example, a Read Only Memory (ROM). The secondary storage unit 230 is, for example, a disc recording medium such as a Hard Disk Drive (HDD), a CD-ROM, a DVD disc, or a Blu-ray disc. The auxiliary storage unit 230 may be a removable medium (a portable storage medium). Examples of the removable medium include a USB memory or an SD card. The above-described recording medium is an example of a storage medium. The communication I/F 240 is, for example, a LAN (Local Area Network) interface board or wireless communication circuitry for wireless communication.

In the prediction server 200, the auxiliary storage unit 230 stores an operating system (OS), various programs, various information tables, and the like. Further, in the prediction server 200, the processor 210 loads the program stored in the auxiliary storage unit 230 into the main storage unit 220 and executes the program, thereby realizing various functions as described later. However, some or all of the functions of the prediction servers 200 may be implemented by hardware circuitry such as ASIC or FPGA. Note that the prediction server 200 is not necessarily realized by a single physical configuration, and may be constituted by a plurality of computers that cooperate with each other.

### User Terminal

The user terminal 400 is a terminal used by the user 40. The user terminal 400 is a computer, a personal digital assistant, or the like used by the user 40. The user 40 is a person who desires to provide information (hereinafter, sometimes referred to as “notification information”) for notifying the user 40 of the status of the exhaust gas. The user terminal 400 of the user 40 receives notification information within a predetermined area after a predetermined timing from the prediction server 200 via the network N1.

### Functional Configuration

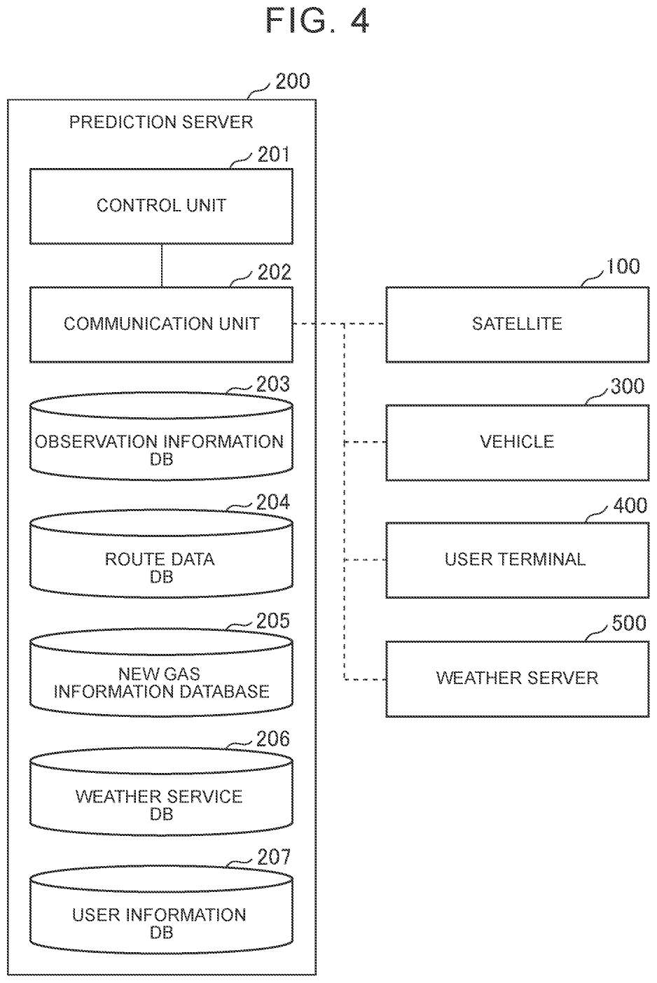

Next, a functional configuration of the prediction server 200 constituting the observation system 1 according to the present embodiment will be described with reference to FIG. 4 to FIG. 9. FIG. 4 is a block diagram schematically illustrating an example of a functional configuration of the prediction server 200 according to the present embodiment. As illustrated in FIG. 4, the prediction server 200 includes a control unit 201, a communication unit 202, an observation information database (observation information DB) 203, a route information database (route information DB) 204, a new gas information database (new gas information DB) 205, a weather information database (weather information DB) 206, and a user information database (user information DB) 207.

The control unit 201 has a function of performing arithmetic processing for controlling the prediction server 200. The control unit 201 can be implemented by the processor 210 in the prediction server 200. The communication unit 202 has a function of connecting the prediction server 200 to the network N1. The communication unit 202 can be realized by a communication I/F 240 in the prediction servers 200.

The observation information DB203 has a function of holding observation information. The observation-information DB203 can be realized by the secondary storage unit 230 in the prediction servers 200. The control unit 201 receives observation information from the artificial satellite 100 by the communication unit 202. The control unit 201 stores the received observation information in the observation information DB203.

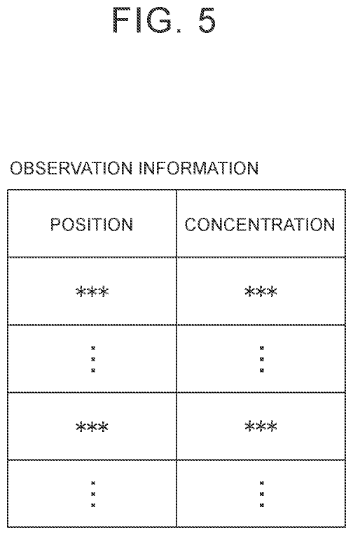

FIG. 5 is a diagram illustrating an exemplary table configuration of observation information held in the observation information DB203. As illustrated in FIG. 5, the observation information includes a position field and a density field. In the position field, information of each position in a predetermined area is input. Specifically, latitude and longitude of each point in a predetermined area are input to the position field. In the concentration field, the concentration of the exhaust gas at a predetermined timing at a point corresponding to the position information input in the position field is input. The control unit 201 acquires the observation information held in the observation information DB203, and thereby can grasp the density of the exhaust gases at the respective positions in the predetermined area at predetermined timings.

The control unit 201 receives, by the communication unit 202, information including the scheduled traveling route from the plurality of vehicles 300. The control unit 201 stores information including the scheduled traveling route received from the plurality of vehicles 300 in the route information DB204. The route information DB204 has a function of storing route information. The route information is information including a travel scheduled route of the plurality of vehicles 300. The route-information DB204 can be realized by the secondary storage unit 230 in the prediction servers 200.

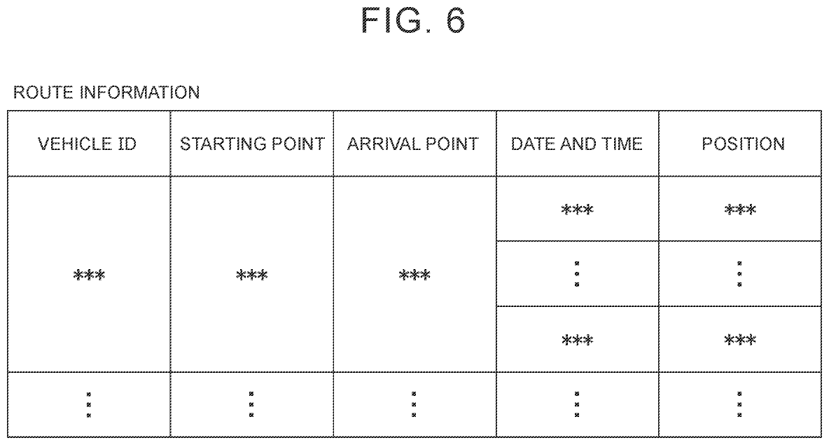

FIG. 6 is a diagram illustrating an exemplary table configuration of route information held in the route information DB204. As shown in FIG. 6, a vehicle ID field, a departure point field, an arrival point field, a date and time field, and a position field are included.

In the vehicle ID field, an identifier (vehicle ID) for specifying the vehicle 300 is input. In the departure point field, information about a point at which the vehicle 300 is scheduled to depart is input on a scheduled traveling route of the vehicle 300 corresponding to the vehicle ID input in the vehicle ID field. In the arrival point field, information about a point at which the vehicle 300 is scheduled to arrive is input on a scheduled traveling route of the vehicle 300 corresponding to the vehicle ID input in the vehicle ID field. In the date and time field, each date and time in a period in which the vehicle 300 corresponding to the vehicle ID input in the vehicle ID field is scheduled to travel is input. In the position field, the position of the vehicle 300 at the date and time input in the date and time field is input.

The new gas information DB205 has a function of holding new gas information. The new gas-information DB205 can be realized by the secondary storage unit 230 in the prediction servers 200. Based on the route information held in the route information DB204, the control unit 201 calculates the traffic volume of the vehicle 300 (the number of vehicles 300 passing through the respective points) at the respective points within the predetermined area after the predetermined timing. Then, the control unit 201 calculates the amount of the new gas based on the traffic volume of the vehicle 300 at each point in the predetermined area after the predetermined timing. Specifically, the control unit 201 multiplies the traffic volume of the vehicle 300 at each point within the predetermined area at each date and time after the predetermined timing by the amount of nitrogen oxides discharged by one vehicle 300 traveling, thereby calculating the amount of exhaust gas (new gas) newly generated at each point at each date and time. Then, the control unit 201 generates new gas information based on the calculated amounts of the new gas at the respective points at the respective dates and times, and stores the new gas information in the new gas information DB205.

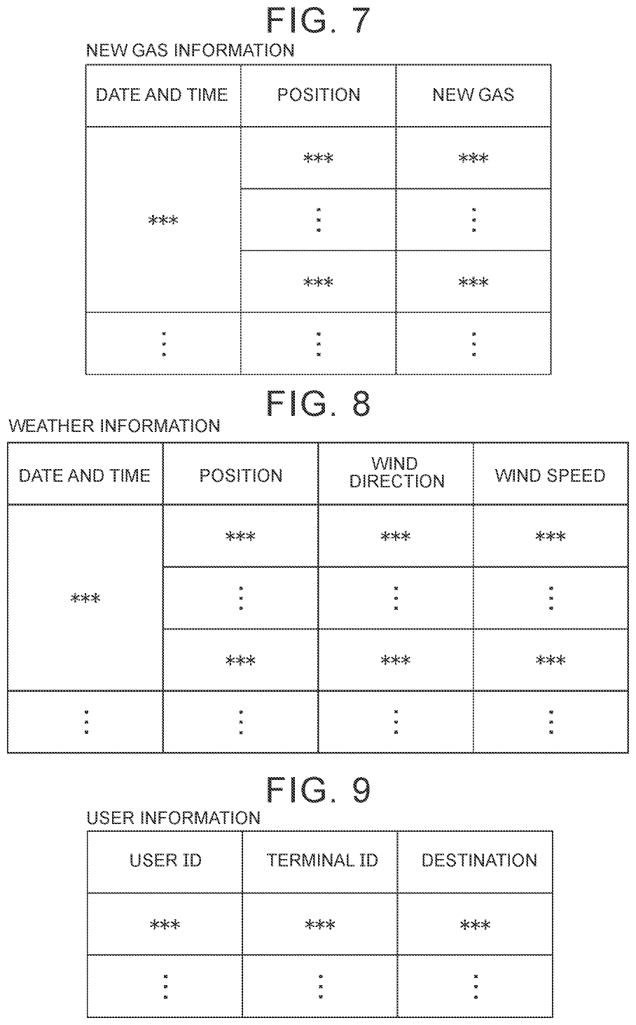

FIG. 7 is a diagram illustrating an exemplary table configuration of new gas information held in the new gas information DB205. As shown in FIG. 7, the new gas information includes a date and time field, a position field, and a new gas field. In the date and time field, each date and time after a predetermined timing is input. In the position field, information indicating each position in a predetermined area is input. Specifically, latitude and longitude of each point in a predetermined area are input to the position field. In the new gas field, the amount of the new gas at each position input in the position field at each date and time input in the date and time field is input.

The control unit 201 receives the weather information from the weather servers 500 by the communication unit 202 and stores the weather information in the weather information DB206. The weather information DB206 has a function of holding weather information. The weather information DB206 can be realized by the secondary storage unit 230 in the prediction servers 200.

FIG. 8 is a diagram illustrating an exemplary table configuration of weather information held in the weather information DB206. As shown in FIG. 8, the weather information includes a date and time field, a position field, a wind direction field, and a wind speed field. In the date and time field, each date and time after a predetermined timing is input in the date and time field. In the position field, information indicating each position in a predetermined area is input. Specifically, latitude and longitude of each point in a predetermined area are input to the position field. In the wind direction field, information on the direction of the wind blowing at each position is input to the date and time input in the date and time field. In the wind speed field, the wind speed of the wind blowing at each position is input at the date and time input in the date and time field.

The control unit 201 acquires the observation information held in the observation information DB203, the new gas information held in the new gas information DB205, and the weather information held in the weather information DB206 from respective databases. The control unit 201 predicts the concentration of the exhaust gas in a predetermined area after a predetermined timing on the basis of the acquired observation information, the new gas information, and the weather information. Specifically, the control unit 201 simulates a state in which the exhaust gas observed in a predetermined area at a predetermined timing is diffused by wind based on the observation information and the weather information. Further, the control unit 201 simulates a state in which the new gas is diffused by the wind based on the new gas information and the weather information. Then, the control unit 201 predicts the transition of the concentration of the exhaust gas at each point in the predetermined area after the predetermined timing based on the state of the diffusion of the exhaust gas observed in the predetermined area at the predetermined timing and the state of the diffusion of the new gas.

The user information DB207 has a function of holding user information. The user information is information related to the user terminal 400 of the user 40. The user information DB207 can be realized by the secondary storage unit 230 of the prediction servers 200.

FIG. 9 is an exemplary table configuration of user information held in the user information DB207. As illustrated in FIG. 9, the user information includes a user ID field, a terminal ID field, and a transmission destination field. In the user ID field, an identifier (user ID) for specifying the user 40 is input. In the terminal ID field, an identifier (terminal ID) for specifying the user terminal 400 of the user 40 is input. In the transmission destination field, information of a transmission destination for transmitting notification information to the user terminal 400 is input. The control unit 201 stores the user information related to the user 40 in the user information DB207 by the user 40 registering the user terminal 400 in the prediction server 200.

The control unit 201 generates notification information regarding the state of the exhaust gas at each point within the predetermined area after the predicted predetermined timing. Here, the notification information includes information on the transition of the concentration of the exhaust gas at each point in the predetermined area after the predetermined timing. Then, the control unit 201 transmits the notification information to the user terminal 400 by the communication unit 202 based on the user information held in the user information DB207.

### (Generation Process)

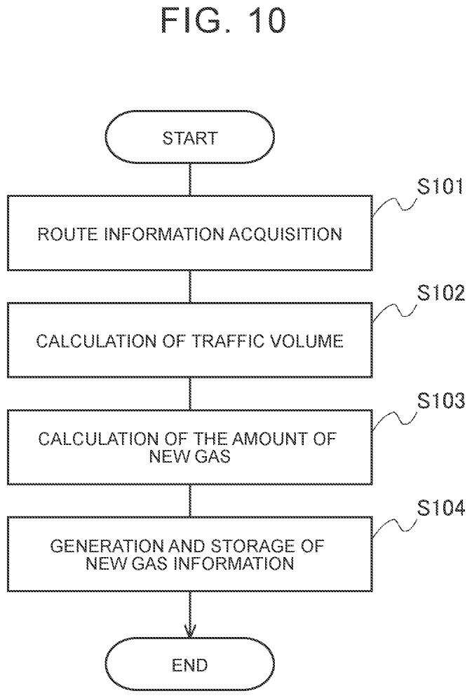

Next, a generation process executed by the control unit 201 in the prediction server 200 in the observation system 1 will be described with reference to FIG. 10. FIG. 10 is a flowchart of a generation process. The generation processing is processing for generating new gas information. The generation process is executed before the notification process described later is executed.

In the generation process, first, in S101, route information is acquired from the route information DB204. Next, in S102, the traffic volume of the vehicles 300 at the respective points in the predetermined area after the predetermined timing is calculated based on the route information. Next, in S103, the amount of new gases is calculated based on the calculated traffic volume. Next, new gas information is generated in S104 and stored in the new gas information DB205. Then, the generation process is ended.

### Notification Process

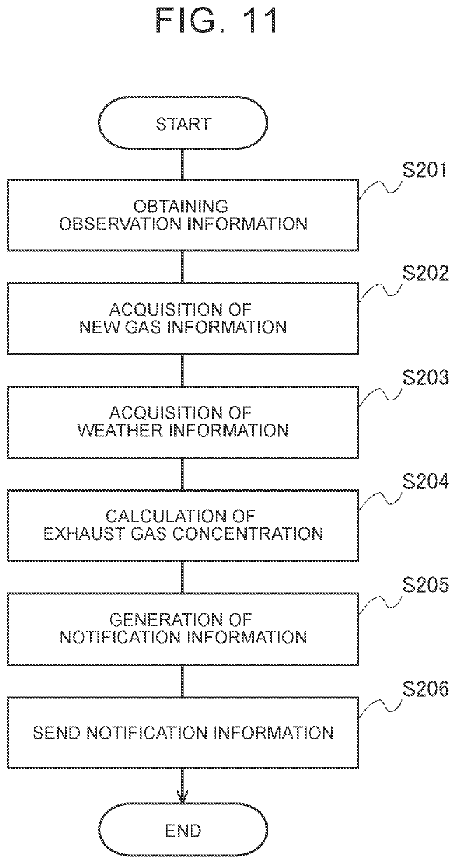

Next, notification processing executed by the control unit 201 in the prediction server 200 in the observation system 1 will be described with reference to FIG. 11. FIG. 11 is a flowchart of notification processing. The notification process is a process for transmitting notification information to the user terminal 400. The notification processing is started after the prediction server 200 receives the observation information from the satellite 100.

In the notification process, first, in S201, observation information is acquired from the observation information DB203. Also, in S202, new gas information is obtained from the new gas information DB205. In addition, in S203, weather information is acquired from the weather information DB206. Next, in S204, based on the observation information, the new gas information, and the weather information, the transition of the density of the exhaust gas at the respective points in the predetermined area after the predetermined timing is predicted. Next, in S205, notification data is generated based on the predicted change in the density of the exhaust gases. Next, in S206, the notification information is transmitted to the user terminal 400 based on the user information held in the user information DB207. Then, the notification process is ended.

As described above, the observation system 1 predicts the state of air pollution in a predetermined area after a predetermined timing. Then, the notification information is transmitted to the user terminal 400. As a result, the user 40 of the user terminal 400 can grasp the state of air pollution in a predetermined area after a predetermined timing. Accordingly, the user 40 can take measures against air pollution, such as refraining from going to a place where the state of air pollution is predicted to deteriorate at a specific time (a place where the concentration of exhaust gas is predicted to increase).

### Modified Example

In the present embodiment, the prediction server 200 calculates the traffic volume of the vehicle 300 within a predetermined area after a predetermined timing on the basis of the information (route information) on the scheduled traveling route of each vehicle 300. However, the prediction server 200 does not necessarily have to calculate the traffic volume of the vehicle 300 based on the route information. For example, the prediction server 200 may acquire a predicted value of the traffic volume of the vehicle 300 at each point within a predetermined area after a predetermined timing from an external device that predicts the traffic volume of the vehicle on the road.

Further, in the present embodiment, the weather information includes information on the wind direction and the wind speed of each point in a predetermined area after a predetermined timing. However, the weather information may include information other than the wind direction and the wind speed of each point in the predetermined area after the predetermined timing, as long as the weather information affects the exhaust gas in the atmosphere. For example, in a case where rain is falling in a predetermined area, it is assumed that the concentration of the exhaust gas is reduced by the exhaust gas in the predetermined area dissolving in the rainwater. Therefore, the weather information may include, for example, information on the amount of precipitation at each point in a predetermined area after a predetermined timing. In this case, the prediction server 200 predicts the concentration of the exhaust gas in the predetermined area after the predetermined timing on the basis of the information on the amount of precipitation at each point in the predetermined area after the predetermined timing.

### Second Embodiment

In the present embodiment, the prediction server 200 makes a proposal to change the scheduled traveling route of the vehicle 300 based on the concentration of the exhaust gas in a predetermined area after a predetermined timing. Hereinafter, differences from the first embodiment will be described.

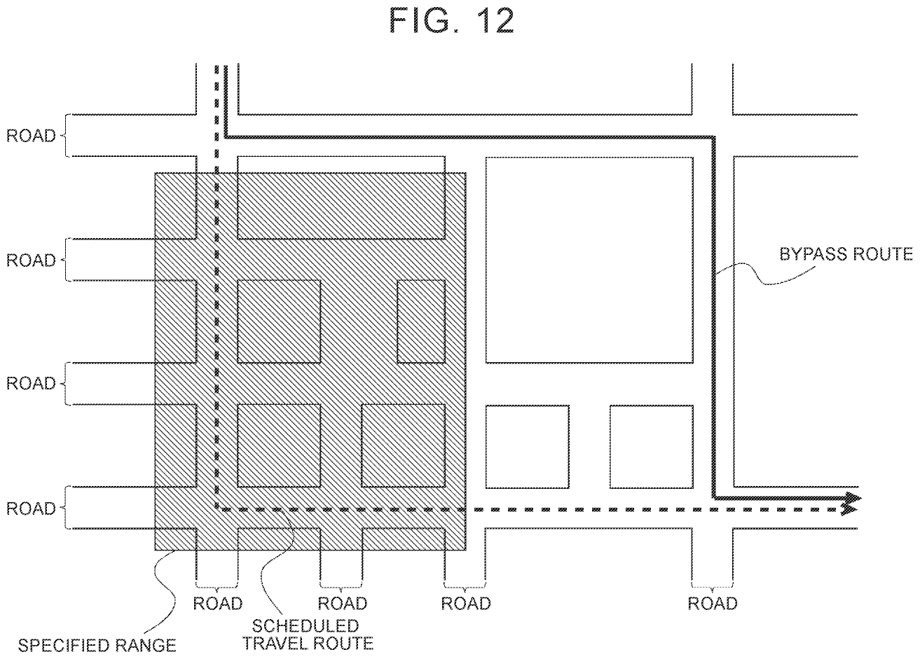

The control unit 201 in the prediction servers 200 acquires the route information held in the route information DB204. Thus, the control unit 201 can grasp where the vehicle 300 traveling within the predetermined area after the predetermined timing is scheduled to travel at each date and time. FIG. 12 is a diagram illustrating an example of a situation of exhaust gas in a predetermined area. In FIG. 12, a scheduled traveling route of the vehicle 300 is indicated by a dotted arrow. Further, in FIG. 12, in a predetermined area, a range (hereinafter, sometimes referred to as a “specific range”) in which the concentration of the exhaust gas is predicted to be equal to or higher than a predetermined level after a predetermined timing is indicated by a hatched portion.

Here, when the vehicle 300 passes through the specific range, it is assumed that the concentration of the exhaust gas in the specific range is higher due to the exhaust gas of the vehicle 300 than when the vehicle 300 does not pass through the specific range. Therefore, the control unit 201 specifies the vehicle 300 (hereinafter, sometimes referred to as “specific vehicle 300”) that is scheduled to pass through the specific range. Then, the control unit 201 transmits information (hereinafter, sometimes referred to as “detour information”) including a travel scheduled route (hereinafter, sometimes referred to as “detour route”) that does not pass through the specific range to the specific vehicle 300. In FIG. 12, the bypass path is indicated by a solid arrow.

Upon receiving the detour information from the prediction server 200, the specific vehicle 300 proposes to change the scheduled traveling route to the detour route to the occupant of the specific vehicle 300. Accordingly, the passenger of the specific vehicle 300 is prompted to prevent the specific vehicle 300 from passing through the specific range.

### (Proposal Processing for Diversion)

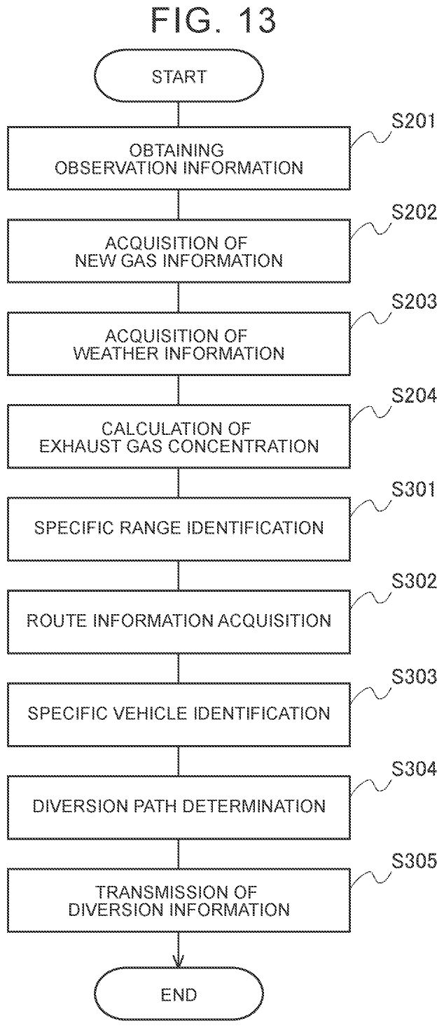

Next, a detour proposal process executed by the control unit 201 in the prediction server 200 in the observation system 1 will be described with reference to FIG. 13. FIG. 13 is a flowchart of the detour proposal process. The detour proposal process is a process for transmitting the detour information to the vehicle 300. Execution of the detour proposal process is started at a predetermined timing. Note that the processing from S201 of the bypass suggestion processing to S204 processing is the same as the processing from S201 to S204 of the notification processing in the first embodiment, and therefore, the explanation thereof is omitted.

In the detour suggestion process, after S204 process is executed, the specified area is specified in S301 based on the density of the exhaust gases in the predetermined area after the predetermined timing. Further, in S302, route information is acquired from the route information DB204. Next, in S303, the specified vehicle 300 is identified based on the information on the scheduled traveling route of the respective vehicles 300 in the route information. Next, in S304, a bypass route for the particular vehicles 300 is determined. Next, in S305, the diversion information is transmitted to the particular vehicle 300. Then, the detour proposal process is ended.

As described above, the observation system 1 predicts the state of air pollution in a predetermined area after a predetermined timing. Then, the detour information is transmitted to the specific vehicle 300. As a result, the passenger of the specific vehicle 300 is prompted to travel on the detour route, and thus the specific vehicle 300 is prevented from passing through the specific range. As a result, it is possible to suppress the concentration of the exhaust gas from becoming high in the specific range due to the specific vehicle 300 passing through the specific range. In this way, it is possible to take measures against air pollution.

### Third Embodiment

In the present embodiment, the price of the fuel of the vehicle in the predetermined area is determined based on the concentration of the exhaust gas in the predetermined area after the predetermined timing. Hereinafter, differences from the first embodiment will be described.

### System Overview

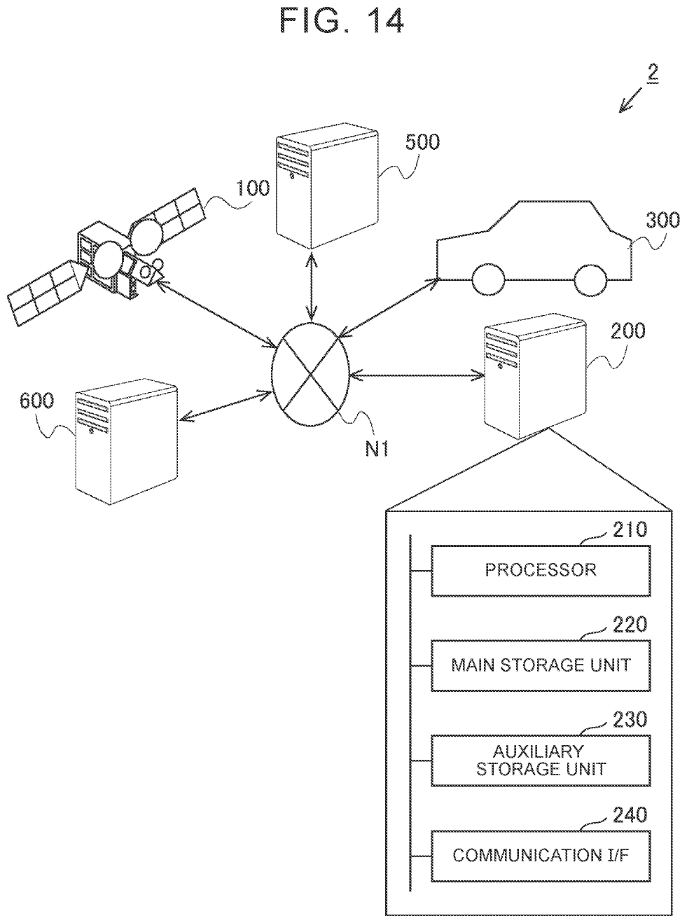

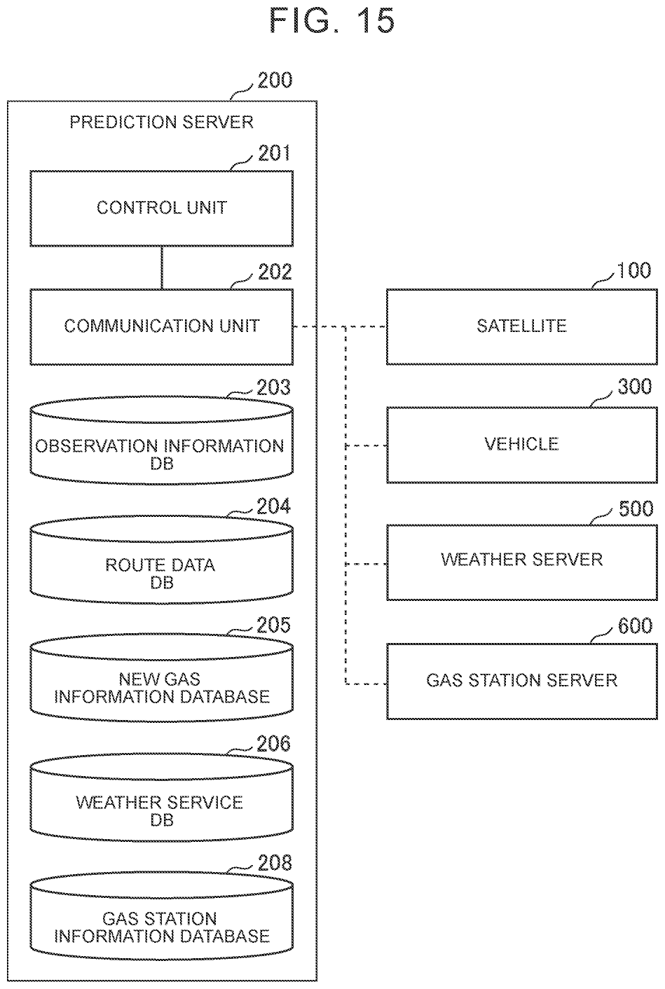

The observation system 2 according to the present embodiment will be described with reference to FIG. 14. FIG. 14 is a diagram illustrating a schematic configuration of the observation system 2 according to the present embodiment. The observation system 2 includes a satellite 100, a prediction server 200, a vehicle 300, a weather server 500, and a gas station server 600. In the observation system 2, a satellite 100, a prediction server 200, a vehicle 300, a weather server 500, and a gas station server 600 are connected to each other via a network N1.

### (Gasoline Station Server)

The gas station server 600 is a server for setting the price of the fuel of the vehicle (hereinafter, sometimes referred to as “fuel price”) in a predetermined area. By changing the fuel price at the gas station server 600, the fuel price sold at each gas station in a predetermined area is changed.

### Functional Configuration

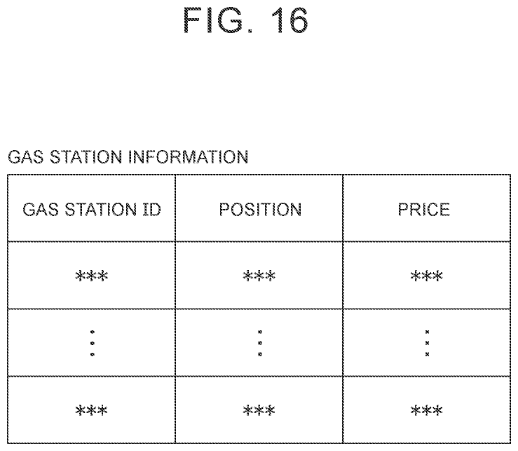

Next, a functional configuration of the prediction server 200 constituting the observation system 2 according to the present embodiment will be described with reference to FIG. 15 and FIG. 16. FIG. 15 is a block diagram schematically illustrating an example of a functional configuration of the prediction server 200 according to the present embodiment. As illustrated in FIG. 15, the prediction server 200 includes a control unit 201, a communication unit 202, an observation information DB203, a route information DB204, a new gas information DB205, a weather information DB206, and a gas station information DB208.

The control unit 201 periodically receives, by the communication unit 202, information related to the fuel price at each gas station in a predetermined area from the gas station server 600. For example, the control unit 201 receives, from the gas station server 600 at a predetermined time every day, information related to the fuel price at each gas station in a predetermined area. The control unit 201 updates the gas station information held in the gas station information DB208 based on the information on the fuel prices of the gas stations in the predetermined area.

The gas station information DB208 has a function of holding gas station information. The gas station information is information about each gas station in a predetermined area. The gas station information DB208 can be realized by the secondary storage unit 230 in the prediction servers 200.

FIG. 16 is a diagram illustrating an exemplary table configuration of the gas station information held in the gas station information DB208. As shown in FIG. 16, the gas station information includes a gas station ID field, a location field, and a price field. In the gas station ID field, an identifier (a gas station ID) for identifying a gas station existing in a predetermined area is input. In the location field, information on a location where the gas station corresponding to the gas station ID input in the gas station ID field exists is input. Specifically, latitude and longitude of each point in a predetermined area are input to the position field.

In the price field, the fuel price in the gas station corresponding to the gas station ID input in the gas station ID field is input. Here, the control unit 201 updates the gas station information by inputting the fuel price of each gas station in the price field on the basis of the information on the fuel price of each gas station in the predetermined area received from the gas station server 600.

The control unit 201 determines the fuel price of each gas station in the predetermined area based on the predicted concentration of the exhaust gas at each point in the predetermined area after the predetermined timing. Specifically, the control unit 201 specifies a predetermined range in which the concentration of the exhaust gas is predicted to be equal to or higher than a predetermined level after a predetermined timing. Here, the predetermined range is a range including each point at which the concentration of the exhaust gas is predicted to be equal to or higher than the predetermined level after the predetermined timing. The control unit 201 specifies, for example, a point at which the concentration of the exhaust gas is predicted to be equal to or higher than a predetermined level for a predetermined time or longer as a point at which the concentration of the exhaust gas is predicted to be equal to or higher than a predetermined level after a predetermined timing.

For example, the control unit 201 specifies, as a predetermined range, an administrative section or the like in a predetermined area including a point at which the concentration of the exhaust gas is predicted to be equal to or higher than a predetermined level after a predetermined timing. Further, for example, the control unit 201 specifies, as a predetermined range, a range within a predetermined distance from a point where the concentration of the exhaust gas is predicted to be equal to or higher than the predetermined level after the predetermined timing. Here, the predetermined distance is a distance at which the exhaust gas of the vehicle 300 is assumed to have an influence on a point at which the concentration of the exhaust gas is predicted to be equal to or higher than a predetermined level after the predetermined timing.

Here, it is assumed that the vehicle 300 travels within a predetermined range, so that the concentration of the exhaust gas within the predetermined range increases as compared with the case where the vehicle 300 does not travel within the predetermined range. Then, the concentration of the exhaust gas at a point where the concentration of the exhaust gas is predicted to be equal to or higher than the predetermined level after the predetermined timing is also increased, and it is assumed that the air pollution situation is deteriorated. Therefore, in the present embodiment, in order to suppress the vehicle 300 from traveling within a predetermined range, the fuel price is changed so that the fuel price in a gas station existing within the predetermined range (hereinafter, sometimes referred to as a “specific gas station”) becomes high. As a result, it is assumed that the fuel price of the specific gas station increases, and thus the occupant of the vehicle 300 is hesitant to drive the vehicle 300 within a predetermined range. As a result, the vehicle 300 is prevented from traveling within a predetermined range.

Specifically, the control unit 201 identifies a gas station existing within a predetermined range as a specific gas station. The control unit 201 determines the fuel price of the specific gas station to be higher than the fuel price of the specific gas station input in the price field in the gas station information. For example, the control unit 201 determines the fuel price of the specific gas station by increasing the fuel price of the specific gas station by a certain ratio. Then, the control unit 201 transmits, by the communication unit 202, price information, which is information indicating the fuel price at the specific gas station, to the gas station server 600. The gas station server 600 changes the price of the gas station in a predetermined area based on the price information received from the prediction server 200.

### (Pricing Process)

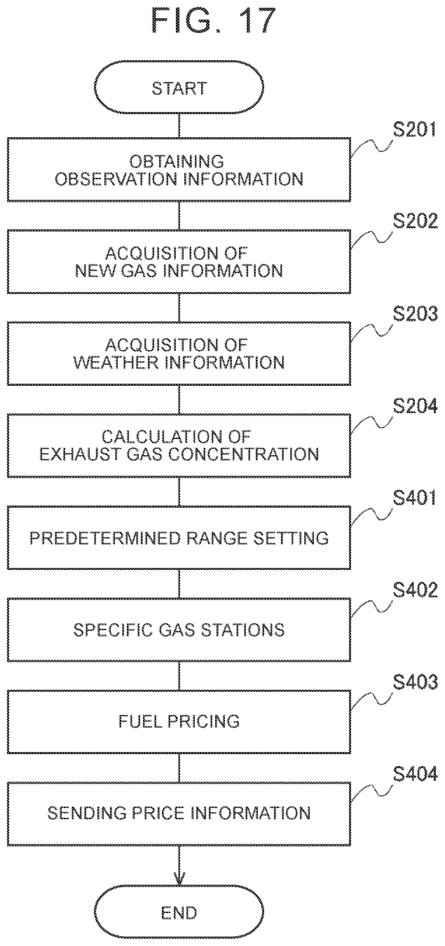

Next, a price determination process executed by the control unit 201 in the prediction server 200 in the observation system 2 will be described with reference to FIG. 17. FIG. 17 is a flowchart of the price determination process. The price determination process is a process for transmitting the price information to the vehicle 300. The execution of the price determination process is started at a predetermined timing. Note that the process from S201 of the pricing process to S204 process is the same as the process from S201 of the notification process to S204 in the first embodiment, and thus the explanation thereof is omitted.

In the pricing process, after S204 process, in S401, a predetermined range including respective points at which the concentration of the exhaust gas is predicted to be equal to or higher than a predetermined level after the predetermined timing is specified based on the predicted concentration of the exhaust gas at respective points within the predetermined area after the predetermined timing. Next, in S402, a particular gas station that is within a predetermined range is identified. Next, in S403, fuel prices at particular gas stations are determined. Next, in S404, the pricing information is transmitted to the gas station server 600. Then, the price determination process is ended.

As described above, the observation system 2 predicts the state of air pollution in a predetermined area after a predetermined timing. Then, the price information is transmitted to the gas station server 600. This increases the fuel price of the specific gas station. At this time, since the fuel price of the specific gas station within the predetermined range increases, the occupant of the vehicle 300 hesitates to drive the vehicle 300 within the predetermined range, and thus the vehicle 300 is prevented from traveling within the predetermined range. As a result, it is possible to suppress the concentration of the exhaust gas from becoming high in a predetermined range by the vehicle 300 traveling within the predetermined range. In this way, it is possible to take measures against air pollution.

### Other Embodiments

The above-described embodiments are mere examples, and the present disclosure can be implemented with appropriate modifications within a range not departing from the scope thereof. Moreover, the processes and units described in the present disclosure can be freely combined and implemented unless technical contradiction occurs.

Further, the processes described as being executed by one device may be shared and executed by a plurality of devices. Alternatively, the processes described as being executed by different devices may be executed by one device. In the computer system, it is possible to flexibly change the hardware configuration (server configuration) for realizing each function.

The present disclosure can also be implemented by supplying a computer with a computer program that implements the functions described in the above embodiments, and causing one or more processors of the computer to read and execute the program. Such a computer program may be provided to the computer by a non-transitory computer-readable storage medium connectable to the system bus of the computer, or may be provided to the computer via a network. Examples of the non-transitory computer-readable storage medium include a random disk (such as a magnetic disk (a floppy disk an HDD, and the like) or an optical disc (such as a CD-ROM, a DVD disc, and a Blu-ray disc)), a ROM, a RAM, an EPROM, an EEPROM, a magnetic card, a flash memory, an optical card, and a random type of medium suitable for storing electronic instructions.

What is claimed is:

1. An information processing device comprising:
a control unit, wherein the control unit executes:
acquisition of first information about exhaust gas in a predetermined area at a predetermined timing observed by an artificial satellite;
acquisition of second information about exhaust gas of a vehicle that is newly generated in the predetermined area at or after the predetermined timing;
acquisition of third information about weather in the predetermined area at or after the predetermined timing; and
prediction of a concentration of the exhaust gas in the predetermined area at or after the predetermined timing based on the first information, the second information, and the third information.

2. The information processing device according to claim 1, wherein the control unit further executes generation of the second information based on a traffic volume of a vehicle that travels in the predetermined area at or after the predetermined timing.

3. The information processing device according to claim 2, wherein the control unit further executes calculation of the traffic volume of the vehicle based on a scheduled traveling route of the vehicle that travels in the predetermined area.

4. The information processing device according to claim 1, wherein the control unit further executes transmission of information about the concentration of the exhaust gas to a terminal associated with a user.

5. The information processing device according to claim 1, wherein the control unit further executes transmission of information including a scheduled traveling route that does not pass through a specific range to a vehicle that is scheduled to pass through the specific range in which the concentration of the exhaust gas is predicted to be a predetermined level or higher.

6. The information processing device according to claim 1, wherein the control unit further executes determination of a price of fuel for the vehicle in the predetermined area based on the concentration of the exhaust gas.

7. The information processing device according to claim 1, wherein the first information is information about a concentration of a substance related to the exhaust gas in the predetermined area at the predetermined timing.

8. An information processing method executed by a computer, comprising:
acquiring first information about exhaust gas in a predetermined area at a predetermined timing observed by an artificial satellite;
acquiring second information about exhaust gas of a vehicle that is newly generated in the predetermined area at or after the predetermined timing;
acquiring third information about weather in the predetermined area at or after the predetermined timing; and
predicting a concentration of the exhaust gas in the predetermined area at or after the predetermined timing based on the first information, the second information, and the third information.

9. The information processing method according to claim 8, further comprising generating the second information based on a traffic volume of a vehicle that travels in the predetermined area at or after the predetermined timing.

10. The information processing method according to claim 9, further comprising calculating the traffic volume of the vehicle based on a scheduled traveling route of the vehicle that travels in the predetermined area.

11. The information processing method according to claim 8, further comprising transmitting information about the concentration of the exhaust gas to a terminal associated with a user.

12. The information processing method according to claim 8, further comprising transmitting information including a scheduled traveling route that does not pass through a specific range to a vehicle that is scheduled to pass through the specific range in which the concentration of the exhaust gas is predicted to be a predetermined level or higher.

13. The information processing method according to claim 8, further comprising determining a price of fuel for the vehicle in the predetermined area based on the concentration of the exhaust gas.

14. The information processing method according to claim 8, wherein the first information is information about a concentration of a substance related to the exhaust gas in the predetermined area at the predetermined timing.

15. A non-transitory storage medium storing instructions executable by one or more processors and causing the one or more processors to perform functions, wherein the functions includes:
acquiring first information about exhaust gas in a predetermined area at a predetermined timing observed by an artificial satellite;
acquiring second information about exhaust gas of a vehicle that is newly generated in the predetermined area at or after the predetermined timing;
acquiring third information about weather in the predetermined area at or after the predetermined timing; and
predicting a concentration of the exhaust gas in the predetermined area at or after the predetermined timing based on the first information, the second information, and the third information.

16. The non-transitory storage medium according to claim 15, wherein the functions further include generating the second information based on a traffic volume of a vehicle that travels in the predetermined area at or after the predetermined timing.

17. The non-transitory storage medium according to claim 16, wherein the functions further include calculating the traffic volume of the vehicle based on a scheduled traveling route of the vehicle that travels in the predetermined area.

18. The non-transitory storage medium according to claim 15, wherein the functions further include transmitting information about the concentration of the exhaust gas to a terminal associated with a user.

19. The non-transitory storage medium according to claim 15, wherein the functions further include transmitting information including a scheduled traveling route that does not pass through a specific range to a vehicle that is scheduled to pass through the specific range in which the concentration of the exhaust gas is predicted to be a predetermined level or higher.

20. The non-transitory storage medium according to claim 15, wherein the functions further include determining a price of fuel for the vehicle in the predetermined area based on the concentration of the exhaust gas.
